(19) United States
(12) Plant Patent Application Publication
(10) Pub. No.: US 2002/0059667 P1
(43) Pub. Date: May 16, 2002

(54) Poinsettia plant named 'Fiselfi'

(75) Inventor: Katharina Zerr, Hohr-Grenzhausen (DE)

Correspondence Address: FOLEY & LARDNER, 3000 K Street, N.W., Suite 500, Washington, DC, 20007-5109, US

(73) Assignee: FLORFIS AG

(21) Appl. No.: 09/850,072

(22) Filed: May 8, 2001

(30) Foreign Application Priority Data
Nov. 11, 2000 (EP) 2000/1675

(51) Int. Cl. A01H 5/00

(52) U.S. Cl. PLT/307

(57) ABSTRACT

A new and distinct cultivar of Poinsettia plant named ‘Fiselfi’, as described and illustrated, and particularly characterized by the combined features of brilliant red bract color; medium sized, star-shaped inflorescence with relatively narrow, pointed bracts; dark-green foliage with distinct lobing and with pointed tips; compact and round plant habit; early flowering response; and good keeping quality after sale.

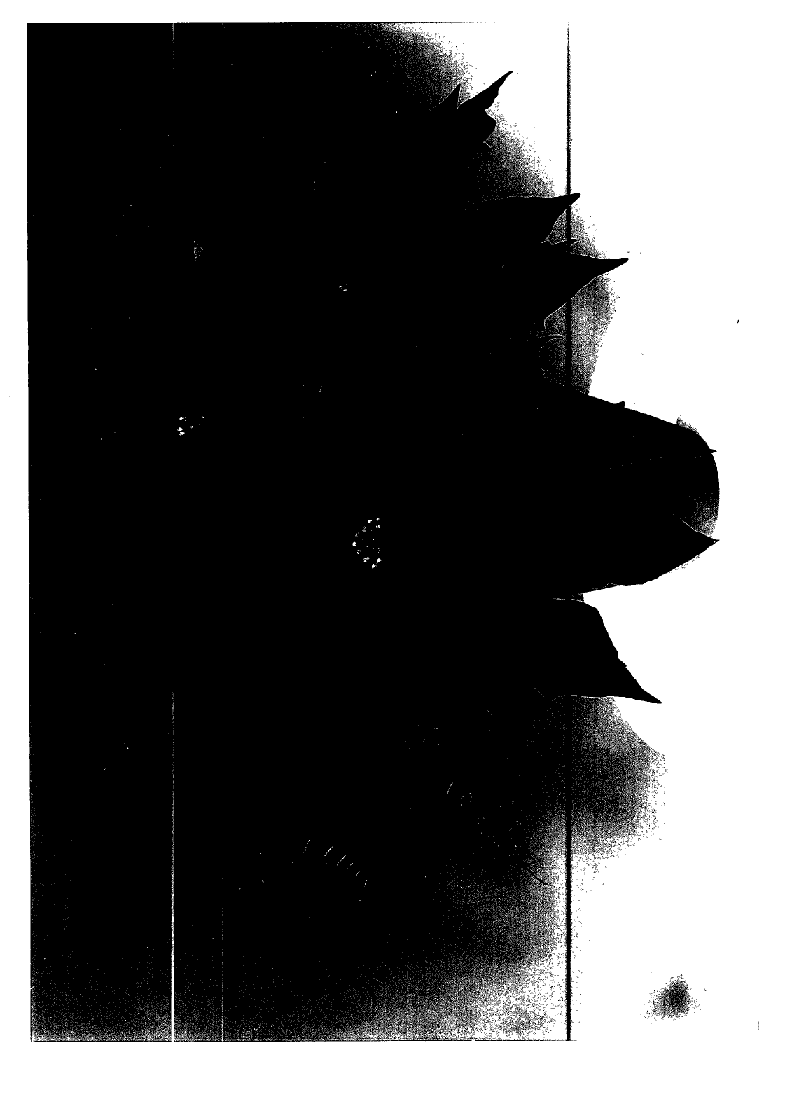

### BACKGROUND OF THE INVENTION

The present invention relates to a new and distinct cultivar of Poinsettia plant known by the cultivar name ‘Fiselfi’, and botanically known as Euphorbia pulcherrima.

‘Fiselfi’ is a product of a planned breeding program which had the objective of creating new Poinsettia cultivars with red flower color in combination with dark-green foliage and good cultivation ability.

The female parental plant was a proprietary hybrid seedling, no. S90-1204-1, characterized by orange-red bract color, medium green foliage, and vigorous growth. The pollen parent is unknown, but is supposed to have been a red flowered plant with dark-green leaves. ‘Fiselfi’ was originated from seeds harvested/plucked by the inventor, Katharina Zerr, in a green-house in Hillscheid, Germany, in 1994.

The seeds germinated in Hillscheid, Germany, from January to February 1995, and the resulting seedlings were identified by numbers. In the summer of 1995, a cutting was taken from each seedling by the breeder. These cuttings were rooted and grown out for examination as flowering single-stem plants from November to December, 1995. ‘Fiselfi’ was discovered and selected as one flowering plant no. 3218, within the progeny of the stated parentage by Katharina Zerr in December 1995. After this plant had been chosen, more cuttings were taken from the original seedling, in 1996, that were grafted on rootstocks of the variety ‘Beckmann's Altrosa’ (U.S. Plant Pat. No. 9,336) in order to transmit the branching causing agent, phytoplasma, into the clone to induce better branching ability. From the upper area of the successfully grafted plants, shoot tip cuttings were taken for the cultivation of branched plants for the second examination in the fall of 1997.

Horticultural examination initiated in the fall of 1997 and continuing thereafter, has confirmed that the combination of characteristics as herein disclosed for ‘Fiselfi’ are firmly fixed and retained through successive generations of asexual reproduction. The new cultivar reproduces true to type.

### BRIEF DESCRIPTION OF THE INVENTION

The following traits have been repeatedly observed and are determined to be basic characteristics of ‘Fiselfi’ which in combination distinguish this Poinsettia as a new and distinct cultivar:

1. brilliant red bract color;

2. medium sized, star-shaped inflorescence with relatively narrow, pointed bracts;

3. dark-green foliage with distinct lobing and with pointed tips;

4. compact and round plant habit;

5. early flowering response; and

6. good keeping quality after sale.

‘Fiselfi’ has not been observed under all possible environmental conditions. The phenotype may vary significantly with variations in environment such as temperature, light intensity, and day length. The following observations, measurements and comparisons describe plants grown in Hillscheid, Germany, under greenhouse conditions which approximate those generally used in commercial practice.

Of the may commercial cultivars known to the inventor, the most similar in comparison to ‘Fiselfi’ are the cultivars Poinsettia Plant named ‘Fismille’ (U.S. Plant patent application Ser. No. 09/210,123), Poinsettia Plant named ‘Fisson’ (U.S. Plant Pat. No. 9,365), and Poinsettia Plant named ‘Fiscor’ (U.S. Plant Pat. No. 9,364).

In comparison to ‘Fismille’, ‘Fiselfi’ has a similar bract color, and begins flowering nearly as early, but in contrast to ‘Fismille’, bracts and leaves of ‘Fiselfi’ develop relatively strong, pointed lobes, and plant habit of ‘Fiselfi’ is much lower and more round in shape.

In comparison to ‘Fisson’, ‘Fiselfi’ is relatively close with respect to bract color and its oak-shaped bracts and leaves. The bracts of ‘Fiselfi’, however appear narrower and are more upright directed, while those of ‘Fisson’ are horizontally or slightly downward directed. Furthermore, Fiselfi is more tolerant to being cultivated at low temperatures, and its plant habit is usually more compact. The differences of growth are best visible in early culture or after plants have been grown at higher temperatures, when ‘Fisson’ grows distinctly more vigorously.

In comparison to ‘Fiscor’, ‘Fiselfi’ has a more brilliantly red bract color, stronger lobing of bracts and leaves, and weaker reddish coloring at the stem. Furthermore, plants are smaller by about 20% when grown under the same cultivation regime.

### BRIEF DESCRIPTION OF THE DRAWING

The accompanying color photographic drawing shows typical inflorescence and foliage of ‘Fiselfi’, with colors being as true as possible with illustrations of this type. The photograph shows a mature potted plant.

### DETAILED BOTANICAL DESCRIPTION

The plants described were grown in a greenhouse in Hillscheid, Germany, in the fall of 1999. Rooted cuttings were planted into 12 cm pots on July 23, and were pinched 12 days after that, leaving 8 nodes. The minimum temperature was about 20° C. until the end of September, and about 17-18° C. thereafter. The plants initiated flowers under natural short-day conditions in fall. Observations and measurements were mainly taken at the beginning of full flowering.

In the following description, color references are made to The Royal Horticultural Society Colour Chart (RHS). The color values were determined indoors in a north light.

Classification:

Botanical.—Euphorbia pulcherrima

Commercial.—Poinsettia cv. ‘Fiselfi’

Parentage: Female Parent: S90-1204-1

Plant:

Form.—Shrub, self-branching

Growth habit.—Compact and round, well-branched

Height (above soil line).—21.0 cm

Width.—38.0 cm

Average number of branches.—7.0

Average number of fully developed inflorescence.5.9

Stem color.—Mainly fresh green RHS 143 A, in parts with slight reddish infusion, approximately RHS 183 D

Rooting.—Fast to medium, less than 20 days

Blooming habit.—Begin under natural short day conditions in fall; botanically (cyathia open) in early December; commercially (bracts colored, marketable) approximately November 20-25

Flowering response time.—about 8 weeks

Flowering season.—Up to 6 weeks

Lasting quality (shelf life).—Very good, 35 days or more

Foliage:

Shape.—Ovate, with acute to obtuse base, with medium to strong, pointed lobes, and with acuminate base

Margin.—Entire

Upper leaf surface.—Smooth and flat texture; only weakly veined, vein color RHS 137 C, red RHS 53 B or lighter, at the base of the midrib

Lower leaf surface.—Flat and smooth texture, except for the slightly protruding midrib and finer side veins, in a palmate pattern, the vein color is very light green, RHS 145 D to light brownish pink, RHS 181 D

Size.—Leaf blade length 12 cm Leaf blade width 9.8 cm

Petiole.—8.0 cm long

Leaf Color.—Uniform, dark-green Mature foliage: Upper surface RHS 139 A; lower surface, RHS 137 B New foliage: Upper surface approximately RHS 143 B; lower surface approximately RHS 144 C Leaf petiole: Upper surface dark red-purple, RHS 59 A to RHS 59 B, lower surface brownish-pink, RHS 180 C

Aspect.—The petioles are horizontally directed, with the leaf blades slightly downward directed

Disease Resistance: no special observations made

Flowering Description:

Whole inflorescence with surrounding bracts: slightly funnel-shaped, outline star-shaped because of the long pointed tips of the bracts, and average diameter 22 cm

Bracts:

Shape.—Narrow ovate, with obtuse bases, pointed lobes, and acuminate tips

Surface Texture/Veination.—Smooth and flat, with the veins hardly visible, the veins are arranged in a palmate pattern similar to the foliage leaves.

As bracts mature, veins create a slightly more rugose pattern similar to the foliage leaves, the vein color of the upper surface corresponds closely to the bract color, the vein color of the lower surface ranges from pink, RHS 54 A to brownish pink, RHS 181 B-C

Size of the largest bract.—Leaf blade 12 cm long, 9.75 cm wide, petiole length is 1.75 cm

Color.—Generally: a brilliant medium red, uniform, no fading near the margins, upper surface RHS: 45 B to 46 B; lower surface RHS: 46 C

Petiole color.—Upper side dark-red RHS 60 A to 185 A, lower side brownish, approximately RHS 181 C, to greenish

Cyme.—Narrow, 15-18 mm diameter

Cyatia.—Borne: about 5-10, in a tight cluster, color grass green RHS 143B, top red, diameter of a single cyathium 5 mm Nectar Cups: golden yellow to orange

Reproductive Organs:

Stamens.—Red filaments, yellow pollen

Pistils.—Red style and stigma, 6-lobed stigma

Ovaries.—Grass-green, RHS 143 A, triangular, 3 ovules

Fruit/Seed set.—Fertile, however under the conditions of the trial, no seed set was observed

I claim:

1. A new and distinct cultivar of Poinsettia plant named ‘Fiselfi’, as described and illustrated herein.
